(19) United States
(12) Patent Application Publication
(10) Pub. No.: US 2026/0033949 A1
(43) Pub. Date: Feb. 5, 2026

(54) DELIVERY APPARATUS FOR A PROSTHETIC IMPLANT

(71) Applicant: EDWARDS LIFESCIENCES CORPORATION, Irvine, CA (US)

(72) Inventors: Kevin Jiang, Irvine, CA (US); Kurt Kelly Reed, Costa Mesa, CA (US)

(21) Appl. No.: 18/790,051

(22) Filed: Jul. 31, 2024

Related U.S. Application Data
(63) Continuation of application No. PCT/US2023/012008, filed on Jan. 31, 2023.
(60) Provisional application No. 63/267,393, filed on Feb. 1, 2022.

(51) Int. Cl. A61F 2/24; A61F 2/958; A61M 25/01

(52) U.S. Cl. CPC A61F 2/2436; A61F 2/958; A61M 25/0136

(57) ABSTRACT

Devices and methods for providing a pathway for fluid to flow around a main lumen of a guide catheter are disclosed. As one example, a delivery apparatus can include a handle including an outer housing and a flush port coupled to the outer housing, a shaft extending distally from the handle, and a main lumen extending axially through the shaft, where the main lumen is fluidly coupled to the flush port via a flush lumen extending between the flush port and the main lumen. The delivery apparatus can further include at least one axially extending channel fluidly coupled to and radially offset from the main lumen, the channel extending between a first location that is adjacent to the flush lumen and a second location that is adjacent to a distal end of the shaft.

### CROSS-REFERENCE TO RELATED APPLICATIONS

This application is a continuation of PCT patent application no. PCT/US2023/012008 filed on Jan. 31, 2023, which claims the benefit of U.S. Provisional Patent Application No. 63/267,393, filed Feb. 1, 2022, each of which is incorporated by reference herein in its entirety.

### FIELD

The present disclosure relates to guide catheters for delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing an implant delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The implant delivery apparatus can be inserted through a lumen of the guide catheter to help direct the implant delivery apparatus to a target implantation site (e.g., a native valve region) within the patient and/or help position the implant delivery apparatus at the target implantation site.

### SUMMARY

Described herein are prosthetic heart valves, docking devices, delivery apparatuses, guide catheters, and methods for implanting docking devices and prosthetic heart valves. The disclosed guide catheters can, for example, be configured to receive a portion of a delivery apparatus within a main lumen of the guide catheter in order to introduce the delivery apparatus into a patient's vasculature and guide the delivery apparatus toward a target implantation site for a prosthetic medical device mounted on the delivery apparatus. In some examples, the guide catheter can include one or more axially extending channels that are fluidly coupled with the main lumen and provide a pathway for fluid to flow around a delivery apparatus being navigated through the main lumen. As such, the devices and methods disclosed herein can, among other things, overcome one or more of the deficiencies of typical guide catheters.

A delivery apparatus for a prosthetic implant can comprise a handle and one or more shafts coupled to the handle.

In some examples, a delivery apparatus can comprise a shaft comprising a main lumen, wherein the main lumen includes an axially extending channel fluidly coupled and radially offset from the main lumen, the channel extending between a flush lumen of the delivery apparatus and a distal end portion of the shaft.

In some examples, a delivery apparatus can comprise a handle including an outer housing and a flush port coupled to the outer housing; a shaft extending distally from the handle; a main lumen extending axially through the shaft, wherein the main lumen is fluidly coupled to the flush port via a flush lumen extending between the flush port and the main lumen; and at least one axially extending channel fluidly coupled to and radially offset from the main lumen. The at least one channel can extend between a first location that is adjacent to the flush lumen and a second location that is adjacent to a distal end of the shaft.

In some examples, a delivery apparatus comprises one or more of the components recited in Examples 1-14 below.

A delivery assembly can comprise an implant catheter and a guide catheter, the guide catheter comprising a handle and a shaft extending distally from the handle and having a main lumen that is configured to receive a portion of the implant catheter therethrough.

In some examples, a delivery assembly can comprise an implant catheter and a guide catheter. The guide catheter can comprise a handle; a shaft extending distally from the handle and having a main lumen that is configured to receive a portion of the implant catheter therethrough; and one or more axially extending channels fluidly coupled to and radially offset from the main lumen Each channel of the one or more axially extending channels can have a first end disposed in the handle and an opposite, second end disposed in a distal end portion of the shaft such that when the implant catheter is arranged within the main lumen, the first end of the channel is disposed proximal to a prosthetic medical device mounted on a distal end portion of the implant catheter and the second end of the channel is disposed distal to the prosthetic medical device.

In some examples, a delivery assembly comprises one or more of the components recited in Examples 15-26 below.

A method for implanting a prosthetic medical device can comprise inserting a shaft of a guide catheter into a vessel of a patient, and inserting a distal end portion of a first implant catheter into a proximal end of the guide catheter and pushing the distal end portion of the first implant catheter through a main lumen of the guide catheter toward a target implantation site for a prosthetic medical device mounted on the distal end portion of the first implant catheter.

In some examples, a method for implanting a prosthetic medical device can comprise inserting a shaft of a guide catheter into a vessel of a patient; inserting a distal end portion of a first implant catheter into a proximal end of the guide catheter and pushing the distal end portion of the first implant catheter through a main lumen of the guide catheter toward a target implantation site for a prosthetic medical device mounted on the distal end portion of the first implant catheter; and during the pushing, flowing fluid through an axially extending channel of the guide catheter that is fluidly coupled with the main lumen such that the fluid flows around the first implant catheter and between a distal end portion and a proximal end portion of the shaft of the guide catheter.

In some examples, a method comprises one or more of the features recited in Examples 27-36, 53 below.

The above method(s) can be performed on a living animal or on a simulation, such as on a cadaver, cadaver heart, anthropomorphic ghost, simulator (e.g., with body parts, heart, tissue, etc. being simulated).

A guide sheath can comprise a shaft having a main lumen defined by an inner surface of a wall of the shaft.

In some examples, a guide sheath can comprise a shaft having a main lumen defined by an inner surface of a wall of the shaft and one or more axially extending channels extending radially outward from the main lumen. Each channel of the one or more axially extending channels depresses into the wall, toward an outer surface of the wall of the shaft.

In some examples, a guide sheath can comprise a shaft having a main lumen defined by an inner surface of a wall of the shaft and an axially extending bypass channel extending through the wall of the shaft and including a first opening into the main lumen that is disposed adjacent to a distal end of the shaft and a second opening into the main lumen that is disposed adjacent to a proximal end of the shaft.

In some examples, a guide sheath comprises one or more of the components recited in Examples 37-52 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a docking device delivery apparatus implanting a docking device for a prosthetic heart valve at a mitral valve of a patient, according to an example.

FIG. 2A schematically illustrates the docking device of FIG. 1 fully implanted at the mitral valve of the patient after the docking device delivery apparatus has been removed from the patient.

FIG. 2B schematically illustrates a prosthetic heart valve delivery apparatus implanting a prosthetic heart valve in the implanted docking device of FIG. 2A at the mitral valve of the patient, according to an example.

FIG. 3 is side view of an exemplary guide catheter configured to receive a delivery apparatus and guide the delivery apparatus through a portion of a patient's vasculature.

FIG. 4 is a cross-sectional side view of the guide catheter of FIG. 3.

FIG. 5 is a perspective view of an exemplary delivery apparatus for a prosthetic heart valve.

FIG. 6 is a side view of a delivery assembly including the guide catheter of FIG. 3 and the delivery apparatus of FIG. 5.

FIG. 7 is a cross-sectional end view of a shaft of a guide catheter which includes a plurality of axially extending channels that extend radially outward from a main lumen of the guide catheter.

FIG. 8A is a first cross-sectional side view of the guide catheter of FIG. 7 showing a portion of the guide catheter with the axially extending channels.

FIG. 8B is a second cross-sectional side view of the guide catheter of FIG. 7 showing a portion of the guide catheter without the axially extending channels.

FIG. 9 is a cross-sectional side view of a guide catheter which includes an axially extending bypass channel off a main lumen of the guide catheter.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Introduction to the Disclosed Technology

As introduced above, a guide catheter can be inserted into a patient's vasculature and then receive an implant delivery apparatus within a main lumen of the guide catheter in order to direct the delivery apparatus therethrough to a target implantation site for a prosthetic implant.

In some examples, an inner diameter of the main lumen of the guide catheter and an outer diameter of portions of the implant delivery apparatus can be closely matched, thereby resulting in decreased space within the main lumen for air and/or blood to pass around the portions of the implant delivery apparatus, and as a result friction between the main lumen and delivery apparatus and/or a vacuum can be created. This can increase push forces felt by a user as they push the implant delivery apparatus through the guide catheter. Accordingly, improvements to the guide catheter that increase space for fluid flow and reduce push forces when advancing an implant delivery apparatus through the guide catheter are desirable.

Described herein are various systems, apparatuses, methods, or the like, that, in some examples, can be used in or with delivery apparatuses for prosthetic medical device (such as prosthetic heart valves or docking devices). In some examples, such systems, apparatuses, and/or methods can provide a path for fluid to flow (e.g., passively flow) within a guide catheter as a prosthetic medical device mounted on a delivery apparatus is navigated through a lumen of the guide catheter toward an implantation site in a body of a patient. The fluid flow path in the guide catheter can reduce vacuum pressure created within the system, thereby reducing push forces felt by a user pushing the delivery apparatus through the guide catheter and increasing an overall efficiency of the system.

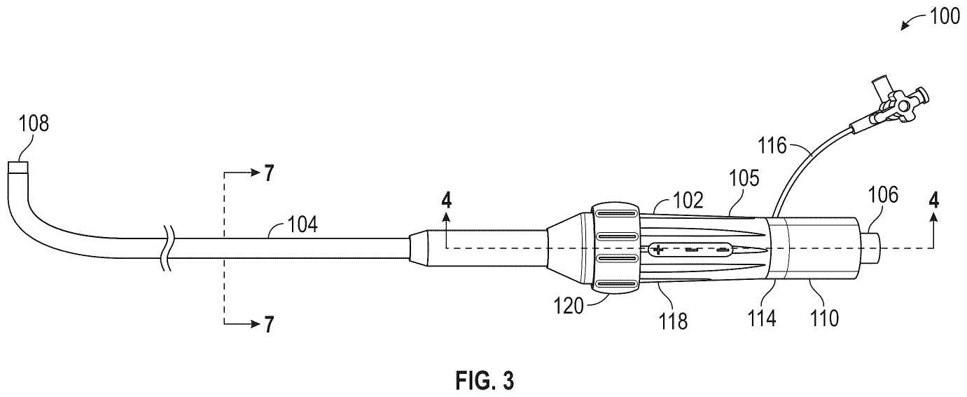

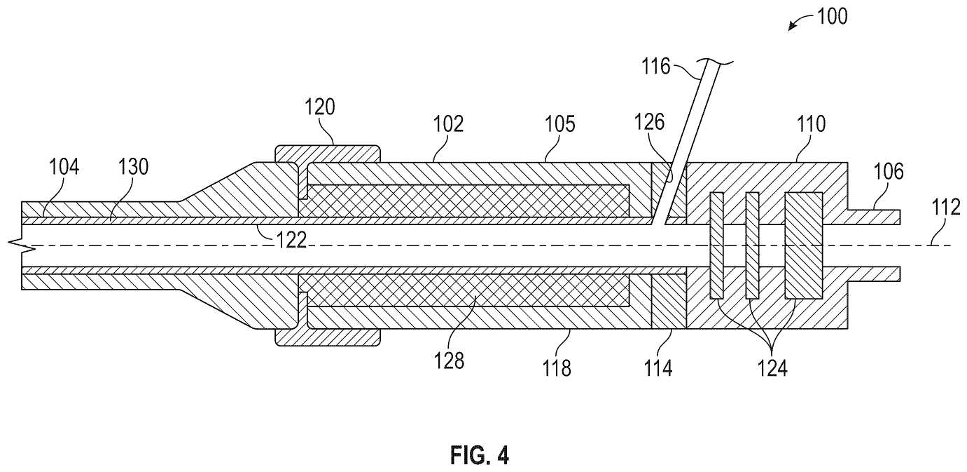

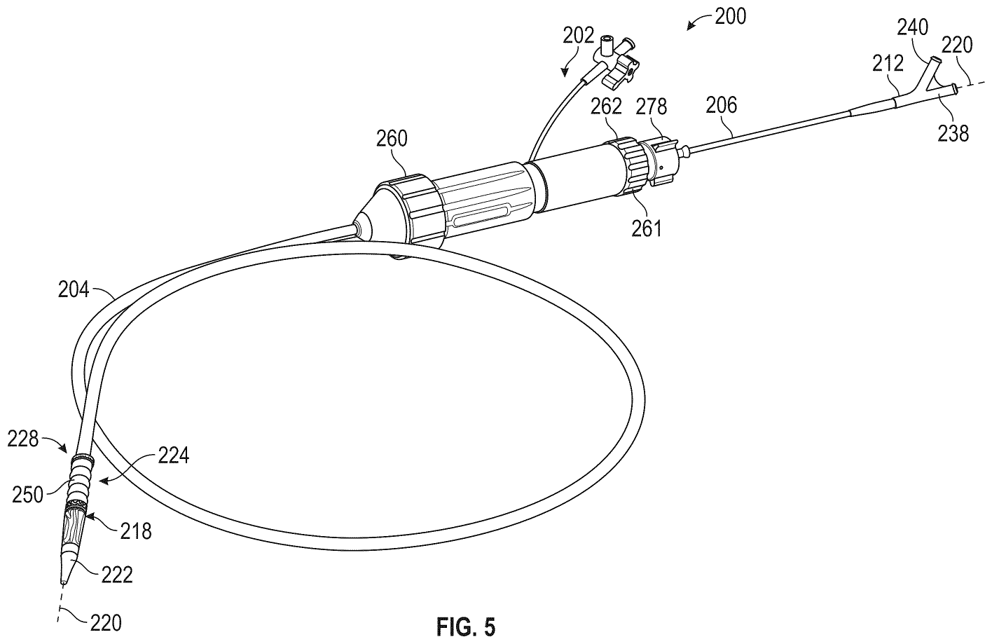

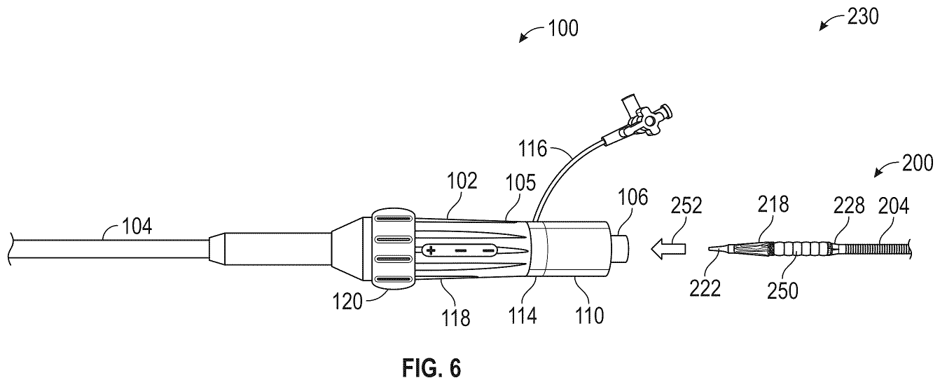

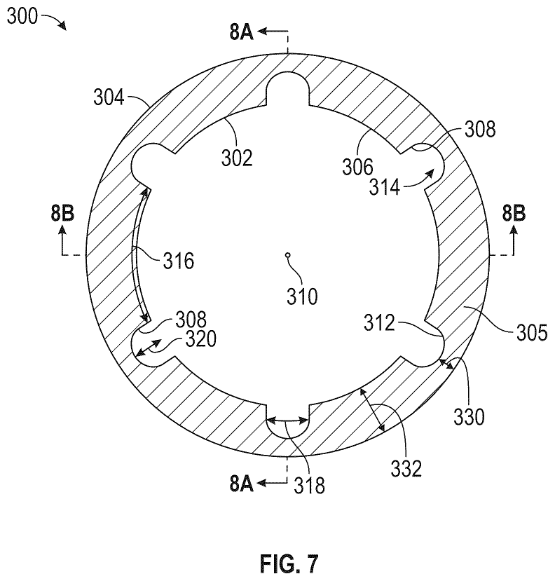

For example, a guide catheter, such as the guide catheter shown in FIGS. 3 and 4, can be inserted into a vessel of a patient and a delivery apparatus (such as that shown in FIG. 5) including prosthetic medical device (e.g., a prosthetic heart valve) mounted thereon can be navigated through a main lumen of the guide catheter toward a target implantation site for the prosthetic medical device, for example a shown in FIG. 6. In some examples, as shown in FIGS. 7-9, the guide catheter can include one or more axially extending channels that are fluidly coupled to and radially offset from the main lumen. As such, the one or more axially extending channels can provide an alternate pathway for air and/or blood to pass through while a delivery apparatus is disposed within the main lumen. As a result, a pressure gradient across one or more seals within a handle of the guide catheter can be reduced, thereby maintaining hemostasis and/or reducing push forces within the guide catheter.

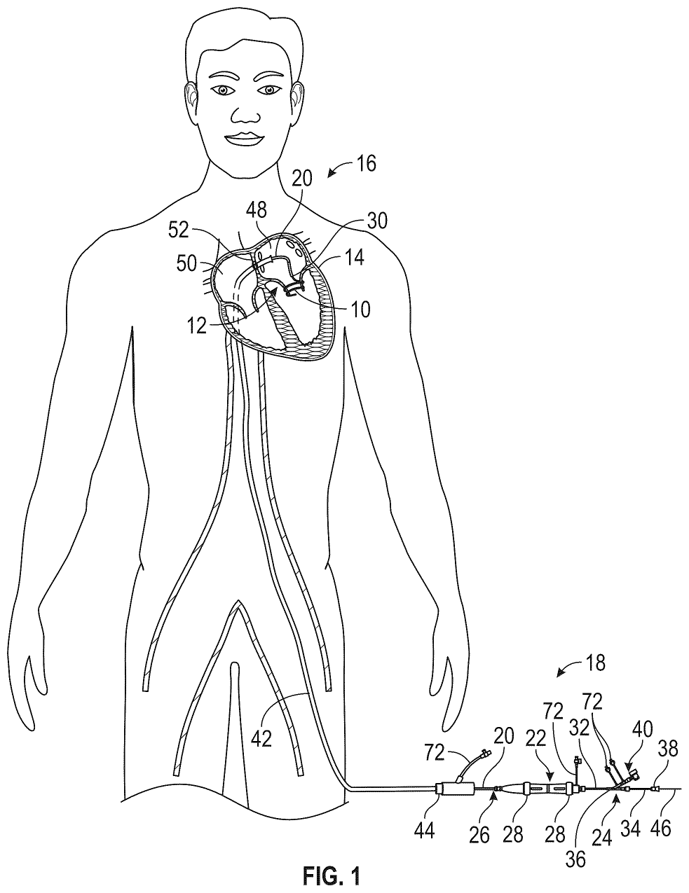

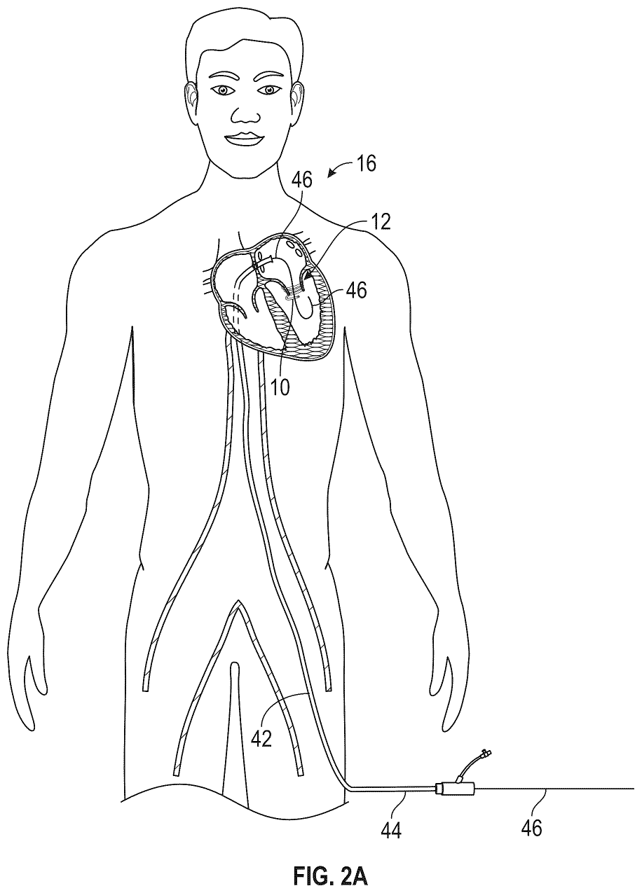

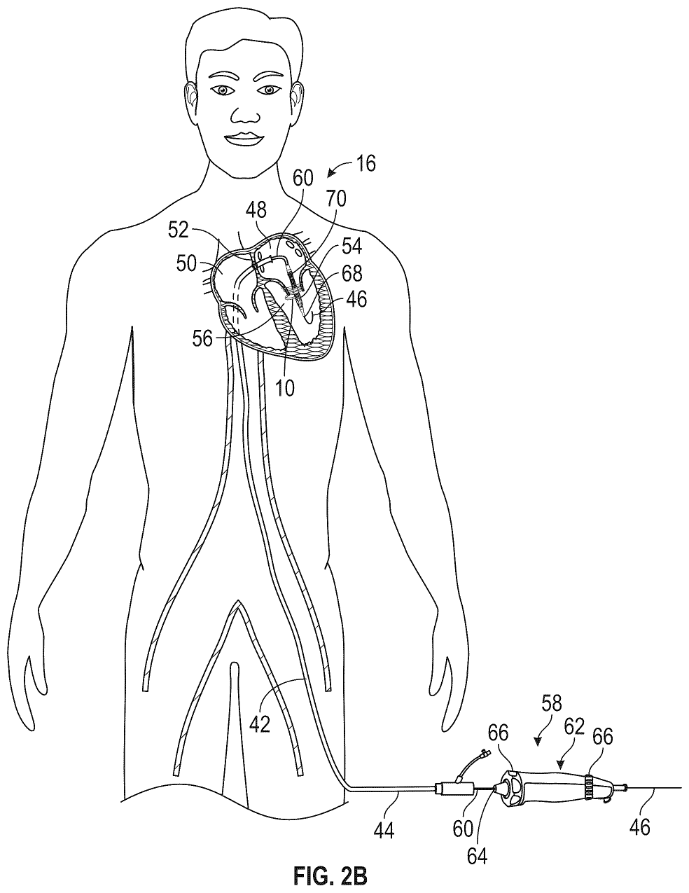

In some examples, the guide catheters disclosed herein can be used to introduce one or more delivery apparatuses (or implant catheters) into the vasculature of a patient and guide the one or more delivery apparatuses at least partially through the vasculature toward a target implantation site. For example, FIGS. 1-2B schematically illustrate an exemplary transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus and then the prosthetic heart valve delivery apparatus is used to deliver a transcatheter prosthetic heart valve (THV) inside the docking device.

As introduced above, defective native heart valves may be replaced with transcatheter prosthetic heart valves (THVs). However, such THVs may not be able to sufficiently secure themselves to the native tissue (e.g., to the leaflets and/or annulus of the native heart valve) and may undesirably shift around relative to the native tissue, leading to paravalvular leakage, valve malfunction, and/or other issues. Thus, a docking device may be implanted first at the native valve annulus and then the THV can be implanted within the docking device to help anchor the THV to the native tissue and provide a seal between the native tissue and the THV.

### Examples of the Disclosed Technology

FIGS. 1-2B depict an exemplary transcatheter heart valve replacement procedure which utilizes a docking device, according to one example. During the procedure, a user first delivers and implants the docking device at a patient's native heart valve using a docking device delivery apparatus (FIG. 1), then removes the docking device delivery apparatus from the patient after implanting the docking device (FIG. 2A), and finally implants the prosthetic valve within the implanted docking device using a prosthetic valve delivery apparatus (FIG. 2B).

FIG. 1 depicts a first stage in an exemplary mitral valve replacement procedure where a docking device 10 is being implanted at a mitral valve 12 of a heart 14 of a patient 16 using a docking device delivery apparatus 18 (which may also be referred to as “catheter” and/or “docking device delivery device”).

In general, the docking device delivery apparatus 18 comprises a delivery shaft 20, a handle 22, and a pusher assembly 24. The delivery shaft 20 is configured to extend into the patient's vasculature and provide a passageway for the docking device 10 to reach the implantation site (e.g., mitral valve 12). Specifically, the delivery shaft 20 may be configured to be advanced through the patient's vasculature to the implantation site by the user and may be configured to receive and/or retain the docking device 10 therein. In some examples, the delivery shaft 20 may comprise an outer sheath or shaft that defines a lumen, and the pusher assembly 24 and/or docking device 10 may be configured to be received and/or advanced within this lumen.

The handle 22 is configured to be gripped and/or otherwise held by the user to advance the delivery shaft 20 through the patient's vasculature. Specifically, the handle 22 is coupled to a proximal end 26 of the delivery shaft 20 and is configured to remain accessible to the user (e.g., outside the patient 16) during the docking device implantation procedure. In this way, the user can advance the delivery shaft 20 through the patient's vasculature by exerting a force on (e.g., pushing) the handle 22. In some examples, the delivery shaft 20 may be configured to carry the pusher assembly 24 and/or docking device 10 with it as it advances through the patient's vasculature. In this way, the docking device 10 and/or pusher assembly 24 may advance through the patient's vasculature in lockstep with the delivery shaft 20 as the user grips the handle 22 and pushes the delivery shaft 20 deeper into the patient's vasculature.

In some examples, the handle 22 may comprise one or more articulation members 28 that are configured to aid in navigating the delivery shaft 20 through the patient's vasculature. Specifically, the articulation members 28 may comprise one or more of knobs, buttons, wheels, and/or other types of physically adjustable control members that are configured to be adjusted by the user to flex, bend, twist, turn, and/or otherwise articulate a distal end 30 of the delivery shaft 20 to aid in navigating the delivery shaft 20 through the patient's vasculature.

The pusher assembly 24 is configured to deploy and/or implant the docking device 10 at the implantation site (e.g., native valve). Specifically, the pusher assembly 24 is configured to be adjusted by the user to advance the docking device 10 through the delivery shaft 20 and push the docking device 10 out of the distal end 30 of the delivery shaft 20. As described above, the pusher assembly 24 may be configured to extend through the delivery shaft 20, within the lumen defined by the outer sheath of the delivery shaft 20. The pusher assembly 24 also may be coupled to the docking device 10 such that the pusher assembly 24 pushes the docking device 10 through and/or out of the delivery shaft 20 as the pusher assembly 24 advances through the delivery shaft 20. Stated slightly differently, because it is retained by, held, and/or otherwise coupled to the pusher assembly 24, the docking device 10 may advance in lockstep with the pusher assembly 24 through and/or out of the delivery shaft 20.

The pusher assembly 24 comprises a pusher shaft 32 and, in some examples, may also include a sleeve shaft 34. The pusher shaft 32 is configured to advance the docking device 10 through the delivery shaft 20 and out of the distal end 30 of the delivery shaft 20, while the sleeve shaft 34, when included, may be configured to cover the docking device 10 within the delivery shaft 20 and while pushing the docking device 10 out of the delivery shaft 20 and positioning the docking device 10 at the implantation site. In some examples, the pusher shaft 32 can be covered by the sleeve shaft 34 and arranged within an outer shaft or connector of a pusher handle (or hub assembly) 36.

In some examples, the pusher assembly 24 may comprise the pusher handle (also referred to as a hub assembly) 36 that is coupled to the pusher shaft 32 and that is configured to be gripped and pushed by the user to translate the pusher shaft 32 axially relative to the delivery shaft 20 (e.g., to push the pusher shaft 32 into and/or out of the distal end 30 of the delivery shaft 20). The sleeve shaft 34 may be configured to be retracted and/or withdrawn from the docking device 10, after positioning the docking device 10 at the implantation site. For example, the pusher assembly 24 may include a sleeve handle 38 that is coupled to the sleeve shaft 34 and is configured to be pulled by a user to retract (e.g., axially move) the sleeve shaft 34 relative to the pusher shaft 32.

The pusher assembly 24 may be removably coupled to the docking device 10 and as such may be configured to release, detach, decouple, and/or otherwise disconnect from the docking device 10 once the docking device 10 has been deployed at the implantation site. As just one example, the pusher assembly 24 (e.g., pusher shaft 32) may be removably coupled to the docking device 10 via a thread, string, yarn, suture, or other suitable material that is tied or sutured to the docking device 10.

In some examples, the pusher assembly 24 comprises a suture lock assembly 40 that is configured receive and/or hold the thread or other suitable material that is coupled to the docking device 10 via the suture. Thus, the thread or other suitable material that forms the suture may extend from the docking device 10, through the pusher assembly 24, to the suture lock assembly 40. The suture lock assembly 40 may also be configured to cut the thread to release, detach, decouple, and/or otherwise disconnect the docking device 10 from the pusher assembly 24. For example, the suture lock assembly 40 may comprise a cutting mechanism that is configured to be adjusted by the user to cut the thread.

Further details of exemplary docking device delivery apparatuses are described in International Publication No. WO2020/247907, which is incorporated by reference herein in its entirety.

Before inserting the docking device delivery apparatus 18 into the vasculature of the patient 16, the user may first make an incision in the patient's body to access a blood vessel 42. For example, in the example illustrated in FIG. 1, the user may make an incision in the patient's groin to access a femoral vein. Thus, in such examples, the blood vessel 42 may be a femoral vein.

After making the incision at the blood vessel 42, the user may insert an introducer device 44 (which can also be referred to herein as a “delivery apparatus”, “guide catheter”, or “guide sheath”), a guidewire 46, and/or other devices (such as an introducer device or transseptal puncture device) through the incision and into the blood vessel 42. The introducer device 44 (which can include an introducer or guide sheath) is configured to facilitate the percutaneous introduction of various implant delivery devices (e.g., the docking device delivery apparatus 18 and the prosthetic heart valve delivery apparatus 58) into and through the blood vessel 42 and may extend through the blood vessel 42 and into the heart 14 but may, in some instances, stop short of the mitral valve 12. The guidewire 46 is configured to guide the delivery apparatuses (e.g., the introducer device 44, docking device delivery apparatus 18, prosthetic valve delivery apparatus 58, catheters, etc.) and their associated devices (e.g., docking device, prosthetic heart valve, etc.) to the implantation site within the heart 14, and thus may extend all the way through the blood vessel 42 and into a left atrium 48 of the heart 14 (FIG. 1).

In some instances, a transseptal puncture device or catheter can be used to initially access the left atrium 48 prior to inserting the guidewire 46 and the introducer device 44. For example, after making the incision to the blood vessel 42, the user may insert a transseptal puncture device through the incision and into the blood vessel 42. The user may guide the transseptal puncture device through the blood vessel 42 and into the heart 14 (e.g., through the femoral vein and into the right atrium 50). The user can then make a small incision in an atrial septum 52 of the heart 14 to allow access to the left atrium 48 from the right atrium 50. The user can insert and advance the guidewire 46 through the transseptal puncture device within the blood vessel 42 and through the incision in the atrial septum 52 into the left atrium 48. Once the guidewire 46 is positioned within the left atrium 48 and/or the left ventricle 56, the transseptal puncture device can be removed from the patient 16. The user can insert the introducer device 44 into the blood vessel 42 and advance the introducer device 44 into the left atrium 48 over the guidewire 46 (e.g., into the position shown in FIG. 1).

In some instances, an additional introducer or introducer device can be inserted through a lumen of the introducer device 44 prior to inserting the introducer device 44 into the blood vessel 42. In some instances, the additional introducer can include a tapered end that extends out a distal tip of the introducer device 44 and that is configured to guide the introducer device 44 into the left atrium 48 over the guidewire 46. Additionally, in some instances the additional introducer can include a proximal end portion that extends out a proximal end of the introducer device 44. Once the introducer device 44 reaches the left atrium 48, the user can remove the additional introducer from inside the introducer device 44 and the patient 16. Thus, only the introducer device 44 and the guidewire 46 remain inside the patient 16. The introducer device 44 is then in position to receive an implant delivery apparatus and help guide it to the left atrium 48, as described further below.

After positioning the guidewire 46 and the introducer device 44 within the left atrium 48, the user may insert the docking device delivery apparatus 18 (e.g., delivery shaft 20) into the patient 16 by advancing the docking device delivery apparatus 18 through the introducer device 44 and over the guidewire 46. The user may continue to advance the docking device delivery apparatus 18 through the patient's vasculature along the guidewire 46 until the docking device delivery apparatus 18 reaches the left atrium 48, as illustrated in FIG. 1. Specifically, the user may advance the delivery shaft 20 of the docking device delivery apparatus 18 by gripping and exerting a force on (e.g., pushing) the handle 22 of the docking device delivery apparatus 18. While advancing the delivery shaft 20 through the patient's vasculature, the user may adjust the one or more articulation members 28 of the handle 22 to navigate the various turns, corners, constrictions, and/or other obstacles in the patient's vasculature.

Once the delivery shaft 20 reaches the left atrium 48, the user may position the distal end 30 of the delivery shaft 20 at and/or near the posteromedial commissure of the mitral valve 12 using the handle 22 (e.g., the articulation members 28). The user may then push the docking device 10 out of the distal end 30 of the delivery shaft 20 with the pusher assembly 24 to deploy and/or implant the docking device 10 at the mitral valve 12. For example, the user may actuate the pusher handle 36 to axially translate the pusher shaft 32, in a distal direction, relative to the delivery shaft 20, such that the docking device 10 (which can be covered by the sleeve shaft 34) is deployed out of the delivery shaft 20 and moved into a desired position at the implantation site.

In some examples, the docking device 10 may be constructed from, formed of, and/or comprise a shape memory material, and as such, may return to its original, pre-formed shape when it exits the delivery shaft 20 and is no longer constrained by the delivery shaft 20. As one example, the docking device 10 may originally be formed as a coil, and thus may wrap around the ventricular side of the leaflets as it exits the delivery shaft 20 and returns to its original coiled configuration.

After pushing the ventricular portion of the docking device 10 (i.e., the portion of the docking device 10 that is configured to be positioned/disposed within a left ventricle 56 and/or on the ventricular side of the mitral valve leaflets), the user may then release the remaining portion of the docking device 10 (the atrial portion of the docking device 10) from the delivery shaft 20 within the left atrium 48. Specifically, the user may retract the delivery shaft 20 relative to the docking device 10, away from the posteromedial commissure of the mitral valve 12. In some examples, the user may maintain the position of the pusher shaft 32 (e.g., by exerting a holding and/or pushing force on the pusher shaft 32) while retracting the delivery shaft 20 so that the delivery shaft 20 withdraws and/or otherwise retracts relative to the docking device 10 and the pusher shaft 32. In this way, the pusher shaft 32 may hold the docking device 10 in place while the user retracts the delivery shaft 20, thereby releasing the docking device 10 from the delivery shaft 20. In some examples, the user may also retract the sleeve shaft 34 from the docking device 10 to uncover the docking device 10, and in some examples, deploy an expandable sleeve of the docking device 10.

After deploying and/or implanting the docking device 10, the user may decouple and/or otherwise disconnect the docking device delivery apparatus 18 from the docking device 10 by, for example, cutting the thread that is sutured to the docking device 10. As just one example, the user may cut the thread with the cutting mechanism of the suture lock assembly 40. Once the docking device 10 is disconnected from the docking device delivery apparatus 18, the user may retract the entire docking device delivery apparatus 18 (the delivery shaft 20, handle 22, and pusher assembly 24) from the patient 16 so that the user can deliver and implant the THV at the mitral valve 12. For example, the docking device 10 and the THV may be delivered on two different, separate delivery apparatuses, and thus the user may need to remove the docking device delivery apparatus 18 from the patient 16 to make room for the THV delivery apparatus. As another example, the user may need to remove the docking device delivery apparatus 18 from the patient 16 to load the THV onto the delivery apparatus. In either example, the user may need to remove the docking device delivery apparatus 18 from the patient 16 before implanting the THV.

FIG. 2A depicts this second stage in the mitral valve replacement procedure, where the docking device 10 has been fully deployed and implanted at the mitral valve 12 and the docking device delivery apparatus 18 (including the delivery shaft 20) has been removed from the patient 16 such that only the guidewire 46 and the introducer device 44 remain inside the patient 16. The introducer device 44 may remain inside the patient 16 to help percutaneously insert the THV and the valve delivery apparatus into the patient 16, while the guidewire 46 may remain within the patient's vasculature to help advance the THV and the valve delivery apparatus through the patient's vasculature. In some examples, the user may advance the guidewire 46 through the mitral valve 12 and into the left ventricle 56 to ensure that the guidewire 46 routes the THV and the valve delivery apparatus all of the way to the mitral valve 12 and into the docking device 10.

As illustrated in FIG. 2A, the docking device 10 may be configured to wrap around the ventricular side of the leaflets of the mitral valve 12 and squeeze the leaflets radially inward (i.e., radially compress the leaflets) to adjust the size and/or shape of the opening between the two leaflets of the mitral valve 12. For example, the docking device 10 may be configured to reduce the size of the opening of the mitral valve 12 and/or to change the shape of the opening to more closely match the cross-sectional shape and/or profile of the THV (e.g., make the opening more circular for a cylindrical THV). By constricting the mitral valve 12 in this manner, the docking device 10 may provide a tighter fit, and thus a better seal, between the THV and the mitral valve 12.

FIG. 2B depicts a third stage in the mitral valve replacement procedure where the user is delivering and/or implanting a prosthetic heart valve 54 (which may also be referred to herein as “heart valve,” “transcatheter prosthetic heart valve” or “THV” for short, “replacement heart valve,” and/or “prosthetic mitral valve”) within the docking device 10 and/or at the mitral valve 12 using a prosthetic heart valve delivery apparatus 58. Thus, the docking device 10 and prosthetic heart valve 54 may be delivered on different delivery apparatuses at different stages in the mitral valve replacement procedure. Specifically, the docking device 10 may be delivered to the mitral valve 12 with the docking device delivery apparatus 18 during the first stage of the mitral valve replacement procedure and the prosthetic heart valve 54 may then be delivered with the prosthetic heart valve delivery apparatus 58.

The prosthetic heart valve delivery apparatus 58 comprises a delivery shaft 60 and a handle 62 coupled to a proximal end 64 of the delivery shaft 60. The delivery shaft 60 is configured to extend into the patient's vasculature to deliver, implant, expand, and/or otherwise deploy the prosthetic heart valve 54 within the docking device 10 at the mitral valve 12. The handle 62 may be similar to the handle 22 of the docking device delivery apparatus 18 and is similarly configured to be gripped and/or otherwise held by the user to advance the delivery shaft 60 through the patient's vasculature.

In some examples, the handle 62 may comprise one or more articulation members 66 that are configured to aid in navigating the delivery shaft 60 through the patient's vasculature. Specifically, the articulation members 66 may comprise one or more of knobs, buttons, wheels, and/or other types of physically adjustable control members that are configured to be adjusted by the user to flex, bend, twist, turn, and/or otherwise articulate a distal end 68 of the delivery shaft 60 to aid in navigating the delivery shaft 60 through the patient's vasculature.

In some examples, the prosthetic heart valve delivery apparatus 58 may comprise an expansion mechanism 70 that is configured to radially expand and deploy the prosthetic heart valve 54. For example, the expansion mechanism 70 may comprise an inflatable balloon that is configured to be inflated to radially expand the prosthetic heart valve 54 within the docking device 10. The expansion mechanism 70 may be included in and/or coupled to the delivery shaft 60 at and/or proximate to the distal end 68 of the delivery shaft 60.

In some examples, the prosthetic heart valve 54 may be self-expanding and may be configured to radially expand on its own without the expansion mechanism 70.

In some examples, the prosthetic heart valve 54 may be mechanically expandable and the prosthetic heart valve delivery apparatus 58 can include one or more mechanical actuators configured to radially expand the prosthetic heart valve 54.

The prosthetic heart valve 54 may be coupled to the delivery shaft 60 at and/or proximate to the distal end 68 of the delivery shaft 60. In examples where the prosthetic heart valve delivery apparatus 58 includes the expansion mechanism 70, the prosthetic heart valve 54 may be mounted on the expansion mechanism 70 in a radially compressed configuration. In some examples, the prosthetic heart valve 54 may be removably coupled to the delivery shaft 60 such that, after the prosthetic heart valve 54 is radially expanded and deployed from the prosthetic heart valve delivery apparatus 58, the prosthetic heart valve delivery apparatus 58 can be retracted away from the implanted prosthetic heart valve 54 and removed from the patient 16.

The prosthetic heart valve 54 is configured to be received and/or retained within the docking device 10. That is, docking device 10 is configured to receive the prosthetic heart valve 54 and help anchor the prosthetic heart valve 54 to the mitral valve 12. The docking device 10 can also be configured to provide a seal between the prosthetic heart valve 54 and the leaflets of the mitral valve to reduce paravalvular leakage around the prosthetic heart valve 54. Specifically, as introduced above, the docking device 10 may initially constrict the leaflets of the mitral valve 12. The prosthetic heart valve 54 may then push the leaflets against the docking device 10 as it radially expands within the docking device 10 (e.g., via inflation of the expansion mechanism 70). Thus, the docking device 10 and the prosthetic heart valve 54 may be configured to sandwich the leaflets of the mitral valve 12 when the prosthetic heart valve 54 is expanded within the docking device 10. In this way, the docking device 10 may provide a seal between the leaflets of the mitral valve 12 and the prosthetic heart valve 54.

In some examples, one or more of the docking device delivery apparatus 18, the prosthetic heart valve delivery apparatus 58, and/or the introducer device 44 may comprise one or more flushing ports 72 (FIG. 1) that are configured to supply flushing fluid to the lumens thereof (e.g., lumens of the delivery shaft 20 of docking device delivery apparatus 18, the delivery shaft 60 of the prosthetic heart valve delivery apparatus 58, and/or the introducer device 44) to prevent and/or reduce the likelihood of blood clot (e.g., thrombus) formation and/or remove air from the apparatus or system.

Like when delivering the docking device 10, the user may insert the prosthetic heart valve delivery apparatus 58 (e.g., delivery shaft 60) into the patient 16 by advancing the prosthetic heart valve delivery apparatus 58 through the introducer device 44 and over the guidewire 46. The user may continue to advance the prosthetic heart valve delivery apparatus 58 along the guidewire 46 (through the patient's vasculature) until the prosthetic heart valve delivery apparatus 58 reaches the mitral valve 12, as illustrated in FIG. 2B. Specifically, the user may advance the delivery shaft 60 of the prosthetic heart valve delivery apparatus 58 by gripping and exerting a force on (e.g., pushing) the handle 62 of the prosthetic heart valve delivery apparatus 58. While advancing the delivery shaft 60 through the patient's vasculature, the user may adjust the one or more articulation members 66 of the handle 62 to navigate the various turns, corners, constrictions, and/or other obstacles in the patient's vasculature.

The user may advance the delivery shaft 60 along the guidewire 46 until the prosthetic heart valve 54 and/or expansion mechanism 70 is/are positioned/disposed within the docking device 10 and/or the mitral valve 12. For example, the user may advance the delivery shaft 60 along the guidewire 46 until the delivery shaft 60 extends through the mitral valve 12, such that the distal end 68 of the delivery shaft 60 is positioned/disposed within the left ventricle 56. Once the prosthetic heart valve 54 is appropriately positioned/disposed within the docking device 10, the user may radially expand the prosthetic heart valve 54, such as with the expansion mechanism 70, to its fully expanded position or configuration. In some examples, the user may lock the prosthetic heart valve 54 in its fully expanded position (e.g., with a locking mechanism) to prevent the valve from collapsing. After expanding and deploying the prosthetic heart valve 54, the user may decouple and/or otherwise disconnect the delivery shaft 60 from the prosthetic heart valve 54 and remove the delivery shaft 60 from the patient.

Although FIGS. 1-2B specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 18, prosthetic heart valve delivery apparatus 58, introducer device 44, and/or guidewire 46), docking devices (e.g., docking device 10), replacement heart valves (e.g., prosthetic heart valve 54), and/or components thereof may be utilized for replacing these other heart valves.

For example, when replacing a native tricuspid valve, the user may also access the right atrium 50 via a femoral vein but may not need to cross the atrial septum 52 into the left atrium 48. Instead, the user may leave the guidewire 46 in the right atrium 50 and perform the same and/or similar docking device implantation process at the tricuspid valve. Specifically, the user may push the docking device 10 out of the delivery shaft 20 around the ventricular side of the tricuspid valve leaflets, release the remaining portion of the docking device 10 from the delivery shaft 20 within the right atrium 50, and then remove the delivery shaft 20 of the docking device delivery apparatus 18 from the patient 16. The user may then advance the guidewire 46 through the tricuspid valve into the right ventricle and perform the same and/or similar prosthetic heart valve implantation process at the tricuspid valve, within the docking device 10. Specifically, the user may advance the delivery shaft 60 of the prosthetic heart valve delivery apparatus 58 through the patient's vasculature along the guidewire 46 until the prosthetic heart valve 54 is positioned/disposed within the docking device 10 and the tricuspid valve. The user may then expand the prosthetic heart valve 54 within the docking device 10 before removing the prosthetic heart valve delivery apparatus 58 from the patient 16.

In some examples, the user may perform the same and/or similar process to replace the aortic valve but may access the aortic valve from the outflow side of the aortic valve via a femoral artery.

Further, although FIGS. 1-2B depict a mitral valve replacement procedure that accesses the mitral valve 12 from the left atrium 48 via the right atrium 50 and femoral vein, it should be appreciated that the mitral valve 12 may alternatively be accessed from the left ventricle 56. For example, the user may access the mitral valve 12 from the left ventricle 56 via the aortic valve by advancing one or more delivery apparatuses through an artery to the aortic valve, and then through the aortic valve into the left ventricle 56.

FIGS. 3 and 4 illustrate an exemplary guide catheter, which is referred to below as a guide sheath 100 (and can also be referred to herein as a “delivery apparatus” or an “introducer device”). In some examples, the guide sheath 100 can be used in lieu of the introducer device 44 in a docking device and/or a prosthetic valve implantation procedure, as described above with reference to FIGS. 1-2B. The guide sheath 100 can be configured to be inserted into a patient's vasculature and receive an implant catheter or delivery apparatus therein in order to introduce the implant catheter into the patient's vasculature and at least partially guide the implant catheter therein to a target implantations site. An exemplary implant catheter for a prosthetic medical device (referred to below as “delivery apparatus 200”) that can be received within the guide sheath 100 is shown in FIGS. 5 and 6, as described further below. Though the guide sheath 100 is described herein as being used with the delivery apparatus 200, the guide sheath 100 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 100 in the illustrated example comprises a handle 102, an elongated shaft 104 extending distally from the handle 102, and a central longitudinal axis 112. The shaft 104 has a main (or primary) lumen 122 that is defined by an inner surface of a wall 130 of the shaft 104 (FIG. 4). The main lumen 122 is configured to receive a delivery apparatus therein (such as any of the prosthetic device delivery apparatuses or implant catheters described herein). In some examples, as shown in FIG. 4, the shaft 104 can extend into the handle 102. Further, in some examples, the main lumen 122 can extend through the handle 102 to an inlet port 106 disposed at a proximal end of the handle 102. Thus, in some examples, an inner surface of a wall of a portion of the handle (e.g., at the proximal end) can further define the main lumen 122. Thus, the main lumen 122 can extend from the inlet port 106 to a distal end 108 of the shaft 104.

The handle 102 can have an outer housing 105 and can further include a seal housing assembly 110 (which can also be referred to as a “seal stack”) which comprises one or more seals 124 contained therein (FIG. 4). The one or more seals 124 of the seal housing assembly 110 can be configured to fluidly seal the main lumen 122 of the guide sheath 100 from the external environment. For example, the one or more seals 124 of the seal housing assembly 110 can be configured to prevent blood from a patient in which the guide sheath 100 is inserted from exiting the guide sheath 100 and prevent air from the environment from entering the guide sheath 100 (e.g., through the inlet port 106). The one or more seals 124 can include a variety of types of seals, such as a duckbill seal, a flapper seal, an umbrella valve, a cross-slit valve, a dome valve, or the like.

The handle 102 can, in some instances, include an adaptor spine 114 disposed adjacent and distal to the seal housing assembly 110. A flush port 116 can be connected to the outer housing 105 at the adaptor spine 114. A flush lumen 126 of the adaptor spine 114 is connected to the flush port 116 and further connects to the main lumen 122 (FIG. 4). The flush port 116 can be configured to receive fluid through a lumen thereof. In this way, the flush port 116 can be fluidly coupled to the main lumen 122 by the flush lumen 126.

The handle 102 can include a steering mechanism configured to adjust the curvature of the distal end portion of the shaft 104 (as such, the shaft 104 can be referred to as a steerable shaft). In the illustrated example, the handle 102 includes a main body portion 118 disposed adjacent and distal to the adaptor spine 114 and an adjustment member, such as the illustrated rotatable knob 120. The main body portion 118 can house internal flex mechanisms 128 of the guide sheath 100 which are operatively coupled to the rotatable knob 120 (FIG. 4). In some examples, the flex mechanisms 128, and thus the knob 120, can be operatively coupled to the proximal end portion of a pull wire. The pull wire can extend distally from the handle 102 through the shaft 104 and have a distal end portion affixed to the shaft 104 at or near the distal end 108 of the shaft 104. Rotating the knob 120 can increase or decrease the tension in the pull wire, thereby adjusting the curvature of the distal end portion of the shaft 104. Further details on steering or flex mechanisms for a delivery apparatus can be found in U.S. Pat. No. 9,339,384, which is incorporated by reference herein.

FIG. 5 illustrates an exemplary prosthetic heart valve delivery apparatus 200 (which can also be referred to here as an “implant catheter”) that can be used to implant an expandable prosthetic heart valve. In some examples, the delivery apparatus 200 is specifically adapted for use in introducing a prosthetic heart valve into a heart. For example, the delivery apparatus 200 can be used in lieu of the prosthetic heart valve delivery apparatus 58 in a prosthetic valve implantation procedure, as described above with reference to FIG. 2B.

The delivery apparatus 200 in the illustrated example of FIG. 5 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. In some examples, the delivery apparatus 200 can further comprise an inner shaft extending distally from the handle 202 coaxially through the intermediate shaft 206 and the outer shaft 204 and proximally from the handle 202 coaxially through the intermediate shaft.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion that extends proximally from a proximal end of the handle 202, to an adaptor 212. The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

In some examples, the intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft can extend distally beyond the distal end portion of the intermediate shaft 206 toward or to a nose cone 222 at a distal end of the delivery apparatus 200.

In some examples, a distal end of the balloon 218 can be coupled to a distal end of the delivery apparatus 200, such as to the nose cone 222 (as shown in FIG. 5), or to an alternate component at the distal end of the delivery apparatus 200 (e.g., a distal shoulder). An intermediate portion of the balloon 218 can overlay a valve mounting portion 224 of a distal end portion of the delivery apparatus 200 and a distal end portion of the balloon 218 (shown in FIG. 4) can overly a distal shoulder of the delivery apparatus 200. As shown in FIG. 5, a prosthetic heart valve 250 can be mounted around the balloon 218, at the valve mounting portion 224 of the delivery apparatus 200, in a radially compressed state. The prosthetic heart valve 250 can be configured to be radially expanded by inflation of the balloon 218 at a native valve annulus, as described above with reference to FIG. 2B.

A balloon shoulder assembly of the delivery apparatus 200, which includes the distal shoulder, is configured to maintain the prosthetic heart valve 250 (or other medical device) at a fixed position on the balloon 218 during delivery through the patient's vasculature.

The outer shaft 204 can include a distal tip portion 228 (best seen in FIG. 6) mounted on its distal end. In some examples, the outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 5) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, as previously incorporated by reference above.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site).

The delivery apparatus 200 can be introduced into a patient's vasculature via a guide catheter, such as the guide sheath 100 of FIGS. 3 and 4. For example, as shown in FIG. 6, to introduce the delivery apparatus 200 (or an alternate implant catheter or delivery apparatus, such as the docking device delivery apparatus 18 of FIG. 1) into the vasculature of a patient, the shaft 104 of the guide sheath 100 can first be inserted into the patient's vasculature and navigated through the vasculature toward a target implantation site for a medical device or implant. The handle 102 of the guide sheath 100 remains outside the patient and can be accessed by a user (e.g., a physician). The distal end portion of the delivery apparatus 200 (e.g., the nose cone 222 and radially compressed prosthetic heart valve 250) can then be inserted into the inlet port 106 of the handle 102 of the guide sheath 100, as indicated by arrow 252 in FIG. 6. The distal end portion of the delivery apparatus 200 is then pushed through the seal housing assembly and into the main lumen 122 of the guide sheath 100. The delivery apparatus 200 can then continue to be pushed through the inner lumen of the shaft 104, toward the implantation site. The assembly shown in FIG. 6 can be referred to as a delivery assembly 230.

In some examples, an inner diameter of the main lumen 122 of the shaft 104 of the guide sheath 100 and an outer diameter of the prosthetic implant mounted on the delivery apparatus, such as the radially compressed prosthetic heart valve 250, can be closely matched. This can cause the prosthetic heart valve (or other prosthetic implant or portion of the delivery apparatus) to be disposed close to or against the wall defining the main lumen 122 of the guide sheath 100 as the delivery apparatus 200 is pushed through the guide sheath 100. In some examples, this can increase forces felt by a user as they push the delivery apparatus 200 through the guide sheath 100 (referred to herein as “push forces”). This can also result in decreased space within the main lumen 122 of the guide sheath 100 for air and/or another fluid (e.g., blood) to pass around portions of the delivery apparatus 200, such as the prosthetic heart valve 250.

Thus, the inventors herein have realized that it is advantageous to provide one or more pathways or channels for fluid to flow around a delivery apparatus within the main lumen of the guide sheath, as the delivery apparatus is pushed through the guide sheath.

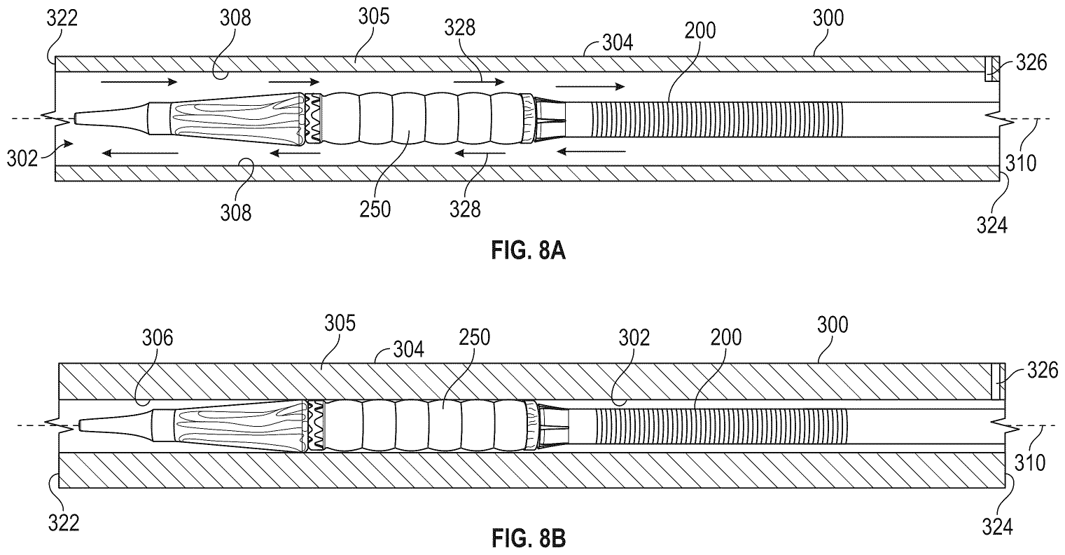

FIGS. 7-8B show an example of shaft 300 for a guide sheath that includes one or more channels configured to allow fluid (e.g., air or blood) to flow around a delivery apparatus or other device disposed within and being pushed through a main lumen 302 (or primary lumen) of the shaft 300. The shaft 300 can be used in lieu of the shaft 104 in the guide sheath 100 of FIG. 3.

As introduced above, the main lumen 302 of the shaft 300 can be defined by a wall 305 of the shaft 300. In some examples, the main lumen 302 can be further defined by a wall of the handle 102. FIG. 7 shows a cross-sectional end view of the shaft 300 (which if used in lieu of the shaft 104 in the guide sheath 100 of FIG. 3 would be taken along the section of the shaft indicated in FIG. 3). The wall 305 of the shaft 300 has an outer surface 304 (e.g., a radially outward facing surface relative to a central longitudinal axis 310 of the shaft 300) and an inner surface 306 (e.g., a radially inward facing surface) that defines the main lumen 302.

FIGS. 8A and 8B show cross-sectional side views of a portion of the shaft 300, taken along the sections indicated in FIG. 7. The sections shown in FIGS. 8A and 8B can include a portion of the shaft 300 that extends from within the handle 102 to distal to the handle 102.

Turning to FIG. 7, the shaft 300 comprises one or more axially extending channels 308 extending radially outward from the main lumen 302 (relative to the central longitudinal axis 310). Each channel 308 can be formed by an inner surface 312, which can be an extension of (e.g., continuous with) the inner surface 306 and that extends radially outward from the inner surface 306 and toward the outer surface 304. In some examples, the inner surface 306 forming the main lumen 302 can be referred to as a first inner surface or inner surface portion and the inner surface 306 forming the channel 308 can be referred to as a second inner surface of inner surface portion.

In this way, each channel 308 can branch off from the main lumen 302 and form a cavity 314 (or space) between (in the radial direction) the inner surface 306 and the outer surface 304. In some examples, the main lumen 302 can be centered along the central longitudinal axis 310 and the one or more channels 308 can be radially offset from the central longitudinal axis 310.

Each channel 308 can depress into the wall 305, toward the outer surface 304, thereby creating thinner wall portions in regions of the channels 308 that have a first thickness 330 that is smaller than a second thickness 332 of portions of the wall 305 disposed between adjacent channels 308. Thus, the wall 305 can have a varying thickness around its circumference.

Though the wall 305 is depicted as a solid wall having a single layer, it should be noted that in some examples the wall 305 can include additional layers, such as one or more reinforcement layers. Further, in some examples, a pull wire or other flex member can extend through the wall 305, thereby allowing a distal end portion of the shaft 300 to be steered through a patient's vasculature, as described above.

In some examples, the shaft 300 can comprise only one channel 308.

In some examples, the shaft 300 can comprise multiple channels 308. Though the shaft 300 is depicted as having six channels 308 in FIG. 7, in alternate examples the shaft 300 can have more or less than six channels 308, such as two, three, four, five, eight, or the like. The number of channels 308 can be even or odd.

The multiple channels 308 can be spaced apart from one another in a circumferential direction, as shown in FIG. 7. A circumferential distance or spacing 316 between adjacent channels 308 can vary in different examples. In some examples, as shown in FIG. 7, the spacing 316 between adjacent channels 308 can be equal for all channels 308 of the shaft 300. In alternate examples, the spacing 316 between adjacent channels 308 can be unequal for at least two pairs of adjacent channels 308 of the shaft 300. For example, in some examples, the shaft 300 can have a first group of multiple channels 308 spaced closer together and a second group of multiple channels 308 spaced closer together, where the first and second groups are spaced farther apart from one another (e.g., such as the first group being disposed at a top or first side of the shaft 300 and the second group being disposed at a bottom or second side of the shaft 300).

Further, a width 318 (in the circumferential direction) and/or depth 320 (in the radial direction) of each channel 308 can vary. In some examples, the width 318 can be selected to be large enough to receive fluid from the main lumen 302 but small enough so that portions of a delivery apparatus or implant catheter passing through the main lumen 302, such as portion of the prosthetic heart valve 250, cannot enter and block the cavity 314 of the channel 308. For example, the width 318 can be 10-35% or 15-30% of the diameter of the main lumen 302.

In some examples, all channels 308 of the shaft 300 can have a same size (e.g., width 318 and depth 320). In alternate examples, at least one channel 308 of the shaft 300 can have a different size than the remaining channels 308 of the of the shaft 300.

The channels 308 can be configured to receive fluid therein, such as air or blood, and allow the fluid to travel axially along the channels 308 while a delivery apparatus 200 (or another implant catheter) is being pushed through the main lumen 302. For example, the channels 308 can allow fluid to flow between a first end 322 of the shaft 300, distal to the prosthetic heart valve 250 mounted on the delivery apparatus 200, to a second end 324 of the shaft 300, proximal or upstream of the prosthetic heart valve 250 (FIGS. 8A and 8B).

As shown in FIG. 8A, each channel 308 can extend axially along the shaft 300, for all or a majority of a length of the shaft 300. For example, each channel 308 can extend from a flush lumen 326 of the shaft 300 (FIG. 8A) toward or to the first end 322 (e.g., distal end) of the shaft 300. The flush lumen 326 can fluidly couple the main lumen 302 with a flush lumen and/or flush port of the handle 102 (e.g., flush port 116 of the handle 102 shown in FIGS. 3 and 4).

FIGS. 8A and 8B illustrate the delivery apparatus 200 within the main lumen 302. The prosthetic heart valve 250 is mounted around the distal end portion of the delivery apparatus 200 and can be pushed through the shaft 300, toward the first end 322. As shown in FIG. 8B which shows a cross-section of the shaft 300 where no channels 308 are present (see FIG. 7), the prosthetic heart valve 250 is disposed proximate, and in some examples flush or brushing against, the inner surface 306 of wall 305 defining the main lumen 302. In contrast, FIG. 8A shows a different cross-section of the shaft 300 where two channels 308 are disposed directly across from one another. As a result, extra space is created for fluid to flow around the prosthetic heart valve 250, as indicated by arrows 328 (FIG. 8A). The arrows 328 illustrate that fluid (e.g., air or blood) can flow in a proximal or distal direction, around the prosthetic heart valve 250. In this way, the channels 308 effectively increase fluid communication within the main lumen 302 of the shaft 300 proximal and distal to the prosthetic heart valve 250, as the prosthetic heart valve 250 travels through the shaft 300 and out its distal end.

It should be noted that the additional space created by the channels 308 shown in FIG. 8A may be exaggerated for the purpose of illustration. In some examples, a distance between the inner surfaces of the channels 308 and the delivery apparatus 200 may be smaller than shown in FIG. 8A.

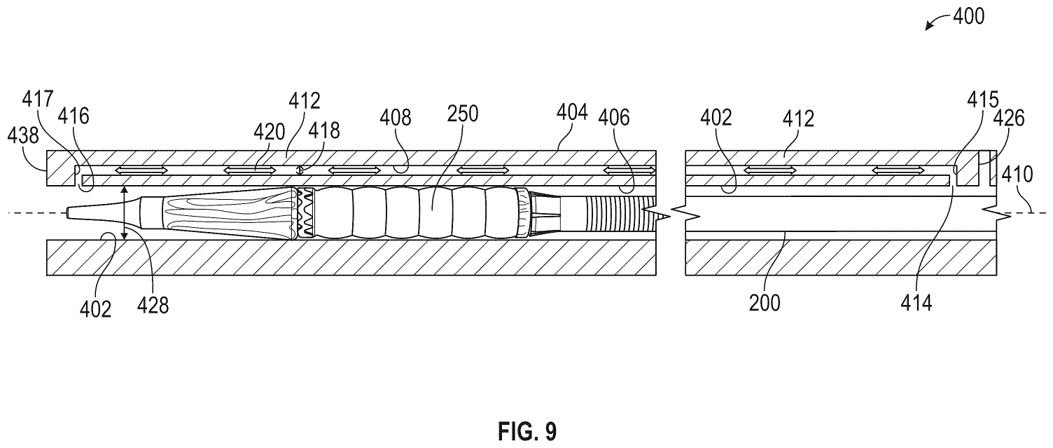

FIG. 9 shows an example of a shaft 400 for a guide sheath that includes one or more channels configured to allow fluid (e.g., air or blood) to flow around a delivery apparatus or other implant catheter disposed within and being pushed through a main lumen 402 of the shaft 400. The shaft 400 can be used in lieu of the shaft 104 in the guide sheath 100 of FIG. 3.

Similar to the shaft 300 of FIGS. 7-8B, the main lumen 402 of the shaft 400 can be defined by an inner surface of a wall 412 of the shaft 400 (and in some examples, also a portion of a wall of the handle 102). The shaft 400 includes an outer surface 404 (e.g., a radially outward facing surface) and an inner surface 406 (e.g., a radially inward facing surface) that defines the main lumen 402. FIG. 9 shows a cross-sectional side view of a distal portion of the shaft 400 and a proximal portion of the shaft 400 that can be disposed within a handle of the guide sheath (e.g., handle 102), where the shaft 400 has a central longitudinal axis 410.

The shaft 400 includes a flush lumen 426 which can fluidly couple the main lumen 402 with a flush lumen and/or flush port of the handle 102 (e.g., flush port 116 shown in FIGS. 3 and 4). For example, the flush lumen 426 extends through a portion of the wall 412 of the shaft 400.

The shaft 400 includes at least one axially extending channel 408 disposed within the wall 412 of the shaft 104. The at least one channel 408 can be a bypass channel with a first opening 414 to the main lumen 402 disposed adjacent to the flush lumen 426 and a second opening 416 to the main lumen 402 spaced apart from the first opening 414 and disposed adjacent to a distal end 438 of the shaft 400. In some examples, a spacing between the distal end 438 and the second opening 416 can be smaller or larger than shown in FIG. 9. However, the spacing can be selected such that the second opening 416 is disposed proximate to the distal end 438 and the second opening 416 remains distal to the prosthetic heart valve 250 until a distal end of the delivery apparatus exits the distal end 438 of the shaft 400.

The channel 408 can be disposed in the wall 412, radially between the inner surface 406 and the outer surface 404. It should be noted that a thickness of the wall 412 may be exaggerated in FIG. 9 for the ease of illustration of the channel 408. Thus, in some examples, the thickness of the wall 412 may be smaller than shown in FIG. 9.

The channel 408 can have various widths or diameters 418 in different examples. In some examples, the width or diameter 418 of the channel 408 can be selected such that fluid can flow through the channel 408, as shown by arrows 420, and reduce push forces felt by a user pushing the delivery apparatus 200 through the shaft 400 to a desired level. As shown in FIG. 9, the width or diameter 418 of the channel 408 is smaller than a diameter 428 of the main lumen 402. In some examples, the width or diameter 418 of the channel 408 can be 10-35% or 15-30% of the diameter 428 of the main lumen 402.

A majority of the channel 408 (apart from the radially extending channel portions that are directly connected to the openings 414 and 416) can be spaced away from the main lumen 402 in the radial direction. As such, the channel 408 can be referred to as a bypass or auxiliary lumen, channel or pathway of the shaft 400 of the guide sheath. For example, the channel 408 can include a first end portion 415 connected to the first opening 414 and a second end portion 417 connected to the second opening 416 and the channel 408 can extend axially along (and through) the wall 412 between the first end portion 415 and the second end portion 417.

In some examples, the shaft 400 can include more than one channel 408, such as two, three, or the like. For example, the shaft 400 could include two channels 408 disposed in the wall 412 and spaced circumferentially apart from one another.

In some examples, a shaft for a guide catheter, such as the guide catheter 100 of FIGS. 3 and 4, can comprise both one or more of the axially extending channels 308 and one or more of the bypass channels 408. For example, a shaft can include both an axially extending channel 308 and a bypass channel 408 extending along its length (or at least a portion of its length, as described above).

In some examples, a proximal portion of the shaft can comprise one or more axially extending bypass channels 408 and a distal portion of the shaft can comprise one or more axially extending channels 308. Alternatively, a proximal portion of the shaft can comprise one or more axially extending channels 308 and a distal portion of the shaft can comprise one or more axially extending bypass channels 408. In this way, different axially extending segments of the same guide catheter shaft can include different types of axially extending channels.

The one or more axially extending channels described above with reference to FIGS. 7-9 can provide one or more pathways for fluid (e.g., air or blood) to passively flow through the guide catheter, around a delivery apparatus or implant catheter being pushed through a main lumen of the guide catheter. In some examples, air can enter the one or more axially extending channels of the guide catheter and flow proximally along the one or more axially extending channels toward a flush port in a handle of the guide catheter. The air can then exit the guide catheter via the flush port. As a result of creating additional pathways for fluid to flow around the main lumen in the guide catheter, a pressure gradient across one or more fluid seals of the handle of the guide catheter (e.g., seals 124 depicted in FIG. 4) can be reduced, thereby increasing a resiliency of the seal housing assembly and maintaining hemostasis within the guide catheter. Additionally, push forces felt by a user pushing a delivery apparatus through the main lumen of the guide catheter can be reduced. In some examples, this can increase an efficiency of a prosthetic device implantation procedure.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a handle including an outer housing and a flush port coupled to the outer housing; a shaft extending distally from the handle; a main lumen extending axially through the shaft, wherein the main lumen is fluidly coupled to the flush port via a flush lumen extending between the flush port and the main lumen; and at least one axially extending channel fluidly coupled to and radially offset from the main lumen, the at least one channel extending between a first location that is adjacent to the flush lumen and a second location that is adjacent to a distal end of the shaft.

Example 2. The delivery apparatus of any example herein, particularly example 1, wherein the handle includes one or more fluid seals disposed within the outer housing, and wherein the flush port is coupled to the outer housing distal to the one or more fluid seals.

Example 3. The delivery apparatus of any example herein, particularly either example 1 or example 2, wherein the shaft extends through a portion of the handle to the first location.

Example 4. The delivery apparatus of any example herein, particularly any one of examples 1-3, wherein the at least one channel extends along an entire length of the shaft.

Example 5. The delivery apparatus of any example herein, particularly any one of examples 1-4, wherein the at least one channel is disposed in a wall of the shaft, and wherein an inner surface of the wall defines the main lumen.

Example 6. The delivery apparatus of any example herein, particularly example 5, wherein the at least one channel extends from the main lumen, radially outward into the wall.

Example 7. The delivery apparatus of any example herein, particularly example 6, wherein a first portion of the wall where the at least one channel is formed therein has a first thickness and a second portion of the wall disposed circumferentially away from the at least one channel has a second thickness, wherein the first thickness is smaller than the second thickness.

Example 8. The delivery apparatus of any example herein, particularly either example 6 or example 7, wherein the at least one channel includes a plurality of axially extending channels spaced apart from one another in a circumferential direction, each channel of the plurality of channels extending radially outward from the main lumen.

Example 9. The delivery apparatus of any example herein, particularly example 8, wherein a thickness of the wall is greater between adjacent channels of the plurality of channels than at each channel.

Example 10. The delivery apparatus of any example herein, particularly example 5, wherein the at least one channel is disposed within the wall and is spaced radially away from the inner surface of the wall except for a first opening and a second opening of the at least one channel into the main lumen.

Example 11. The delivery apparatus of any example herein, particularly example 10, wherein the first opening is disposed at the first location and the second opening is disposed at the second location, and wherein the second location is spaced away from the distal end of the shaft.

Example 12. The delivery apparatus of any example herein, particularly any one of examples 1-11, wherein the main lumen extends from a proximal end of the handle to a distal end of the shaft.

Example 13. The delivery apparatus of any example herein, particularly any one of examples 1-12, wherein the handle further includes a main body portion disposed distal to the flush port, and wherein the main body portion contains flex mechanisms that are configured to adjust a curvature of a distal end portion of the shaft.

Example 14. The delivery apparatus of any example herein, particularly example 13, wherein the handle further includes a rotatable knob operatively coupled to the flex mechanisms.

Example 15. A delivery assembly comprising: an implant catheter; and a guide catheter comprising: a handle; a shaft extending distally from the handle and having a main lumen that is configured to receive a portion of the implant catheter therethrough; and one or more axially extending channels fluidly coupled to and radially offset from the main lumen, wherein each channel of the one or more axially extending channels has a first end disposed in the handle and an opposite, second end disposed in a distal end portion of the shaft such that when the implant catheter is arranged within the main lumen, the first end of the channel is disposed proximal to a prosthetic medical device mounted on a distal end portion of the implant catheter and the second end of the channel is disposed distal to the prosthetic medical device.

Example 16. The delivery assembly of any example herein, particularly example 15, wherein the handle comprises one or more fluid seals in a proximal end portion of the handle and a flush port disposed distal to the one or more fluid seals.

Example 17. The delivery assembly of any example herein, particularly example 16, wherein the handle comprises a flush lumen extending between the flush port and the main lumen, and wherein the first end of each channel is disposed adjacent to the flush port.

Example 18. The delivery assembly of any example herein, particularly example 17, wherein the second end of each channel is disposed adjacent to a distal end of the shaft.

Example 19. The delivery assembly of any example herein, particularly any one of examples 15-18, wherein each channel extends radially outward from the main lumen and depresses into a wall of the shaft, and wherein a first inner surface of the wall defines the main lumen.

Example 20. The delivery assembly of any example herein, particularly example 19, wherein a second inner surface of the wall defining each channel is continuous with the first inner surface of the wall that defines the main lumen.

Example 21. The delivery assembly of any example herein, particularly either example 19 or example 20, wherein the one or more axially extending channels includes a plurality of axially extending channels spaced circumferentially apart from one another around the main lumen, and wherein the wall has a first thickness between adjacent channels of the plurality of axially extending channels and a second thickness at each channel, wherein the first thickness is greater than the second thickness.

Example 22. The delivery assembly of any example herein, particularly any one of examples 15-18, wherein each channel is configured as a bypass channel that extends through a wall of the shaft, and wherein an inner surface of the wall defines the main lumen.

Example 23. The delivery assembly of any example herein, particularly example 22, wherein the first end of each channel includes a first opening into the main lumen and the second end of each channel includes a second opening in the main lumen.

Example 24. The delivery assembly of any example herein, particularly any one of examples 15-23, wherein the implant catheter is configured to deliver a docking device mounted around the distal end portion of the implant catheter.

Example 25. The delivery assembly of any example herein, particularly any one of examples 15-23, wherein the implant catheter is configured to deliver a prosthetic heart valve mounted around the distal end portion of the implant catheter.

Example 26. The delivery assembly of any example herein, particularly any one of examples 15-25, wherein the shaft is a steerable shaft, and wherein the handle comprises a flex mechanism configured to adjust a curvature of a distal end portion of the shaft.

Example 27. A method for implanting a prosthetic medical device, comprising: inserting a shaft of a guide catheter into a vessel of a patient; inserting a distal end portion of a first implant catheter into a proximal end of the guide catheter and pushing the distal end portion of the first implant catheter through a main lumen of the guide catheter toward a target implantation site for a prosthetic medical device mounted on the distal end portion of the first implant catheter; and during the pushing, flowing fluid through an axially extending channel of the guide catheter that is fluidly coupled with the main lumen such that the fluid flows around the first implant catheter and between a distal end portion and a proximal end portion of the shaft of the guide catheter.

Example 28. The method of any example herein, particularly example 27, wherein flowing fluid through the axially extending channel includes allowing one or more of blood and air to passively flow through the axially extending channel, radially outward of the main lumen.

Example 29. The method of any example herein, particularly either example 27 or example 28, wherein the main lumen is defined by an inner surface of a wall of the shaft of the guide catheter, and wherein the shaft of the guide catheter extends into a handle of the guide catheter which remains exterior to the patient while a portion of the shaft extending distally from the handle is disposed within the vessel.

Example 30. The method of any example herein, particularly example 29, wherein the handle includes a flush port disposed distal to one or more fluid seals of the handle that are disposed adjacent to the proximal end of the guide catheter, and wherein the axially extending channel has an end disposed proximate to the flush port such that air flows through the axially extending channel and out the flush port.

Example 31. The method of any example herein, particularly any one of examples 27-30, further comprising implanting the prosthetic medical device at the target implantation site, removing the first implant catheter from the guide catheter, and inserting a second implant catheter into the guide catheter and pushing the second implant catheter through the main lumen toward the target implantation site.

Example 32. The method of any example herein, particularly example 31, wherein the first implant catheter is a docking device delivery apparatus and the prosthetic medical device is a docking device, and wherein the second implant catheter is a prosthetic heart valve delivery apparatus configured to deliver a prosthetic heart valve within the implanted docking device.

Example 33. The method of any example herein, particularly any one of examples 27-32, wherein the axially extending channel extends radially from the main lumen and extends axially along the main lumen.

Example 34. The method of any example herein, particularly example 33, wherein the axially extending channel is a first axially extending channel, and wherein the method further comprises flowing fluid through a second axially extending channel of the guide catheter that extends radially from the main lumen and extends axially along the main lumen, and that is spaced apart from the first axially extending channel in a circumferential direction.

Example 35. The method of any example herein, particularly any one of examples 27-32, wherein the axially extending channel is a bypass channel that extends through a wall of the shaft of the guide catheter and around a portion of the main lumen, and wherein an inner surface of the wall defines the main lumen.

Example 36. The method of any example herein, particularly example 35, wherein the portion of the main lumen is a majority of a length of the main lumen, and wherein the axially extending channel includes a first opening to the main lumen disposed at the proximal end portion of the shaft and a second opening to the main lumen disposed at the distal end portion of the shaft.

Example 37. A guide sheath comprising: a shaft having a main lumen defined by an inner surface of a wall of the shaft; and one or more axially extending channels extending radially outward from the main lumen, wherein each channel of the one or more axially extending channels depresses into the wall, toward an outer surface of the wall of the shaft.

Example 38. The guide sheath of any example herein, particularly example 37, wherein each channel extends along an entire length of the shaft.

Example 39. The guide sheath of any example herein, particularly either example 37 or example 38, wherein a thickness of the wall varies in a circumferential direction, with the thickness of the wall being smallest where each channel is formed therein.

Example 40. The guide sheath of any example herein, particularly any one of examples 37-39, wherein each channel is fluidly coupled with the main lumen along an entire length of the channel.

Example 41. The guide sheath of any example herein, particularly any one of examples 37-40, wherein a width of each channel, in a circumferential direction, is 15-30% of a diameter of the main lumen.

Example 42. The guide sheath of any example herein, particularly any one of examples 37-41, wherein the one or more axially extending channels includes a plurality of axially extending channels that are spaced circumferentially apart from one another around the main lumen.

Example 43. The guide sheath of any example herein, particularly any one of examples 37-42, further comprising a handle, and wherein the shaft extends distally from the handle.

Example 44. The guide sheath of any example herein, particularly example 43, wherein the shaft extends into the handle toward a flush port of the handle, and wherein each channel has an end disposed adjacent to a flush lumen that extends from the main lumen to the flush port.

Example 45. The guide sheath of any example herein, particularly example 44, wherein the handle includes a plurality of fluid seals configured to prevent fluid flow past the plurality of fluid seals, and wherein the flush lumen is disposed distal to the plurality of fluid seals.

Example 46. A guide sheath comprising: a shaft having a main lumen defined by an inner surface of a wall of the shaft; and an axially extending bypass channel extending through the wall of the shaft and including a first opening into the main lumen that is disposed adjacent to a distal end of the shaft and a second opening into the main lumen that is disposed adjacent to a proximal end of the shaft.

Example 47. The guide sheath of any example herein, particularly example 46, wherein the bypass channel extends axially along the shaft, in parallel to the main lumen.

Example 48. The guide sheath of any example herein, particularly either example 46 or example 47, wherein a first diameter of the bypass channel is 15-30% of a second diameter of the main lumen.

Example 49. The guide sheath of any example herein, particularly any one of examples 46-48, wherein the bypass channel is spaced radially away from the inner surface of the wall and extends axially through the wall from a first end portion of the bypass channel that is connected to the first opening and second end portion of the bypass channel that is connected to the second opening.

Example 50. The guide sheath of any example herein, particularly any one of examples 46-49, further comprising a handle, and wherein the shaft extends distally from the handle.

Example 51. The guide sheath of any example herein, particularly example 50, wherein the shaft extends into the handle toward a flush port of the handle, and wherein the second opening of the bypass channel is disposed adjacent to a flush lumen that extends from the main lumen to the flush port.

Example 52. The guide sheath of any example herein, particularly example 51, wherein the handle includes a plurality of fluid seals configured to prevent fluid flow past the plurality of fluid seals, and wherein the flush lumen is disposed distal to the plurality of fluid seals.

Example 53. A method comprising sterilizing the prosthetic heart valve, apparatus, guide sheath, and/or assembly of any example.

The features described herein with regard to any example can be combined with other features described in any one or more of the other examples, unless otherwise stated. For example, any one or more of the features of one guide catheter can be combined with any one or more features of another guide catheter. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus comprising:
a handle including an outer housing and a flush port coupled to the outer housing;
a shaft extending distally from the handle;
a main lumen extending axially through the shaft, wherein the main lumen is fluidly coupled to the flush port via a flush lumen extending between the flush port and the main lumen; and
at least one axially extending channel fluidly coupled to and radially offset from the main lumen, the at least one channel extending between a first location that is adjacent to the flush lumen and a second location that is adjacent to a distal end of the shaft.

2. The delivery apparatus of claim 1, wherein the handle includes one or more fluid seals disposed within the outer housing, and wherein the flush port is coupled to the outer housing distal to the one or more fluid seals.

3. The delivery apparatus of claim 1, wherein the shaft extends through a portion of the handle to the first location.

4. The delivery apparatus of claim 1, wherein the at least one channel extends along an entire length of the shaft.

5. The delivery apparatus of claim 1, wherein the at least one channel is disposed in a wall of the shaft, and wherein an inner surface of the wall defines the main lumen.

6. The delivery apparatus of claim 5, wherein the at least one channel extends from the main lumen, radially outward into the wall.

7. The delivery apparatus of claim 5, wherein the at least one channel includes a plurality of axially extending channels spaced apart from one another in a circumferential direction, each channel of the plurality of channels extending radially outward from the main lumen.

8. The delivery apparatus of claim 7, wherein a thickness of the wall is greater between adjacent channels of the plurality of channels than at each channel.

9. The delivery apparatus of claim 5, wherein the at least one channel is disposed within the wall and is spaced radially away from the inner surface of the wall except for a first opening and a second opening of the at least one channel into the main lumen.

10. The delivery apparatus of claim 9, wherein the first opening is disposed at the first location and the second opening is disposed at the second location, and wherein the second location is spaced away from the distal end of the shaft.

11. A delivery assembly comprising:
an implant catheter; and
a guide catheter comprising:
a handle;
a shaft extending distally from the handle and having a main lumen that is configured to receive a portion of the implant catheter therethrough; and
one or more axially extending channels fluidly coupled to and radially offset from the main lumen, wherein each channel of the one or more axially extending channels has a first end disposed in the handle and an opposite, second end disposed in a distal end portion of the shaft such that when the implant catheter is arranged within the main lumen, the first end of the channel is disposed proximal to a prosthetic medical device mounted on a distal end portion of the implant catheter and the second end of the channel is disposed distal to the prosthetic medical device.

12. The delivery assembly of claim 11, wherein the handle comprises one or more fluid seals in a proximal end portion of the handle and a flush port disposed distal to the one or more fluid seals.

13. The delivery assembly of claim 12, wherein the handle comprises a flush lumen extending between the flush port and the main lumen, and wherein the first end of each channel is disposed adjacent to the flush port.

14. The delivery assembly of claim 11, wherein each channel extends radially outward from the main lumen and depresses into a wall of the shaft, and wherein a first inner surface of the wall defines the main lumen.

15. The delivery assembly of claim 11, wherein each channel is configured as a bypass channel that extends through a wall of the shaft, and wherein an inner surface of the wall defines the main lumen.

16. The delivery assembly of claim 15, wherein the first end of each channel includes a first opening into the main lumen and the second end of each channel includes a second opening in the main lumen.

17. The delivery assembly of claim 11, wherein the implant catheter is configured to deliver a docking device mounted around the distal end portion of the implant catheter.

18. The delivery assembly of claim 11, wherein the implant catheter is configured to deliver a prosthetic heart valve mounted around the distal end portion of the implant catheter.

19. A guide sheath comprising:
a shaft having a main lumen defined by an inner surface of a wall of the shaft; and
one or more axially extending channels extending radially outward from the main lumen, wherein each channel of the one or more axially extending channels depresses into the wall, toward an outer surface of the wall of the shaft.

20. The guide sheath of claim 19, wherein each channel extends along an entire length of the shaft.
